(19) United States
(12) Patent Application Publication
(10) Pub. No.: US 2012/0234320 A1
(43) Pub. Date: Sep. 20, 2012

(54) Metered Dose Inhaler Port for Ventilated Patients

(76) Inventors: Zachary W. O'Ferrell, Sandy, UT (US); John A. Brewer, Marietta, GA (US); William A. Hagen, Canton, GA (US); Angela G.D. Mitchell, Atlanta, GA (US); Cassandra E. Morris, Roswell, GA (US)

(21) Appl. No.: 13/155,915

(22) Filed: Jun. 8, 2011

Related U.S. Application Data
(60) Provisional application No. 61/453,740, filed on Mar. 17, 2011.

(51) Int. Cl. A61M 16/10

(52) U.S. Cl. 128/203.12

(57) ABSTRACT

There is provided a metered dose inhaler port for a ventilated patient where the port has a critical distance (as defined herein) sufficient to allow a metered dose inhaler to discharge into the port when the metered dose inhaler has a dose counter attached to it. The port must also have a stiffness sufficient to avoid bending upon insertion of the metered dose inhaler stem. The disclosure discusses an elongation of the metered dose inhaler actuator port to allow for use with metered dose inhaler canisters with and without dose counters. This solution maintains the original drug path length and so minimizes changes that might change the drug dispersal characteristics of the port and require governmental approval or re-education of medical personnel.

This application claims the benefit of priority from U.S. Provisional Application No. 61/453,740 filed on Mar. 17, 2011 in the names of Zachary W. O'Ferrell, John A. Brewer, William A. Hagen, Angela G. D. Mitchell, and Cassandra E. Morris, the contents of which are incorporated herein by reference.

### BACKGROUND

The present disclosure relates to an improved port for the installation of medicaments for patients being mechanically ventilated.

A metered-dose inhaler (MDI) is a device that delivers a specific amount of medication to the lungs in the form of a short burst of aerosolized medicine that is inhaled by the patient. It is commonly used delivery system for treating asthma, chronic obstructive pulmonary disease (COPD) and other respiratory diseases. The medication in a metered dose inhaler is most commonly a bronchodilator, corticosteroid or a combination of both for the treatment of asthma and COPD. The metered dose inhaler device typically includes a canister for the medicine and a stem through which the medicine is discharged to the patient. Such devices are commonly seen in schools or sports fields where children use inhalers to control asthma. Hand held dispensers hold the metered dose inhaler device within a plastic “boot” that has a large opening that is placed in the mouth. The user may then press the metered dose inhaler canister into the boot, discharging the medicine into mouth at the same time as the user inhales. The metered dose inhaler canister and boot combination will not function, however, for intubated respiratory patients.

Respiratory patient care is a dynamically developing field in medicine, ranging in its needs from infants to the aged. The range of respiratory ailments, both temporary and permanent, to which such patients are subjected are many and varied. For example, the range of procedures for intubated patients may include the following: ventilation, aspiration, oxygenation, sampling, visual inspection, in-line sensing, pressure monitoring, flushing, medicating and/or lavage. Most problems now center or focus on multiple needs of the patient and accommodation of multiple treatments, some to be performed at the same time. The lack of equipment to easily, efficiently, and safely accomplish the multiple therapies in the best interest of the patient has been and continues to be a concern.

In “closed” system suctioning, for example that disclosed in commonly owned U.S. Pat. No. 4,569,344, a device which may be used to suction secretions from the upper airway in intubated patients is enclosed within a generally cylindrical plastic bag to eliminate or minimize contamination of the suction catheter prior to use. This is generally referred to as a “closed suction catheter” device and is available under the trade name TRACH CARE® from BALLARD® Medical Products (Kimberly-Clark Corporation) or KIMVENT®. As the patient requires artificial removal of secretions, the suction catheter may be advanced through one end of the plastic bag, through a connecting fitting or manifold and into the tracheal tube. The other, proximal end of the suction catheter is attached to a source of suction. Closed suction systems are generally preferred by healthcare providers since the provider is better protected from the patient's secretions. Closed suction systems are also easier and quicker to use since a sterile field need not be created each time the patient must be suctioned, as is required in open suction systems. The closed suction catheter may be permanently attached to the proximal end of the tracheal tube or may be detachably connected so that it may be replaced periodically.

In order to provide medicine to a patient that is using a closed suction system, a port allowing access to the air line from the ventilator to the patient is provided. Manifolds that couple to a tracheal tube, a ventilator, and a suction catheter assembly provide a plurality of openings or ports. U.S. patent application Ser. No. 12/347,422, for example, provides a rotary manifold that permits multiple accesses for additional objects, devices or instruments, and reduces or removes the need to open a closed ventilating system. Such a manifold may also include a metered dose inhaler port.

Metered dose inhaler devices used in conjunction with artificial airways and closed suction catheters have been actuated via introduction of the stem of the metered dose inhaler into a metered dose inhaler actuator port in the ventilator line or manifold. A boot is not used. When the actuator is squeezed together (into the port), the stem of the metered dose inhaler is pressed into the metered dose inhaler port and medication is delivered. Recent changes by pharmaceutical companies to the metered dose inhaler canister and stem have rendered current metered dose inhaler actuator ports ineffective. Pharmaceutical companies have added a dose counter that keeps track of the number of doses in the metered dose inhaler canister so that the user knows how much is left in the canister. This dose counter increases the length of the metered dose inhaler canister such that the stem does not reach into the actuator port. Actuation of the metered dose inhaler to discharge the medicine using a prior art port can no longer occur.

One skilled in the art can recognize that, though the improvement in the metered dose inhaler device that allows one to keep track of doses dispensed and remaining is a useful one, it is important that the new design function properly with the intake manifold of the TRACH CARE® device so that the metered dose inhaler stem may be inserted into the metered dose inhaler port and actuated.

### SUMMARY

The problem discussed above has found a solution to a large degree in the present disclosure, which describes the elongation of the metered dose inhaler actuator port to allow for use with metered dose inhaler canisters with and without dose counters. This solution maintains the original drug path length and so minimizes changes that might change the drug dispersal characteristics of the port and require governmental approval or re-education of medical personnel.

In another aspect, the disclosed metered dose inhaler actuator port may have a method for advancing the dose counter within the metered dose inhaler dose counter. This allows a user to track the number of doses that have been dispensed and so anticipate when a new container will be needed.

In another aspect, the disclosed metered dose inhaler actuator port may have a prong for advancing a dose counting wheel within the metered dose inhaler dose counter. This allows a user to track the number of doses that have been dispensed and so anticipate when a new container will be needed.

In another aspect, disclosed is a metered dose inhaler port for a ventilated patient where the port has a critical distance (as defined herein) sufficient to allow a metered dose inhaler to discharge into the port when the metered dose inhaler has a dose counter attached to it.

In another aspect, the port has a stiffness sufficient to avoid bending upon insertion of the metered dose inhaler stem. The port desirably has a stiffness or durometer of greater than 72 A or more desirably at least 85 A as defined using test ASTM D2240.

The port is desirably made from polyvinyl chloride, though other suitable polymers may be used if they are sufficiently stiff. It is desirable that the polyvinyl chloride be phthalate free.

Also provided is a ventilator manifold having a metered dose inhaler port, where the port has a critical distance of 13.46 mm plus or minus 10 percent and a durometer of greater than 72 A or more desirably 85 A plus or minus 10 percent. The port desirably has a critical distance at least 50 percent greater than a prior art device and a Shore Hardness durometer of greater than 72 A or more desirably at least 85 A.

### BRIEF DESCRIPTION OF THE DRAWINGS

FIG. 1 shows a multiple access port manifold of the type described in U.S. patent application Ser. No. 12/347,422.

FIG. 2 shows two versions of the metered dose inhaler device; one without a dose counter (on the left) and one with a dose counter (on the right).

FIG. 3 shows a closer view of the two versions of the metered dose inhaler device; one without a dose counter (on the left) and one with a dose counter (on the right).

FIG. 4 is a cross-sectional view of a metered dose inhaler stem inserted into the prior art metered dose inhaler port.

FIG. 5 shows a cross-sectional view of a metered dose inhaler stem inserted into the disclosed metered dose inhaler port.

FIG. 5A shows a perspective view of a metered dose inhaler port having a prong with teeth that is used to advance a wheel dose counter within the metered dose inhaler dose counter to keep a total of the number of doses dispensed.

FIG. 6 shows another view of a metered dose inhaler stem inserted into the prior art metered dose inhaler port.

FIG. 7 shows another view of a metered dose inhaler stem inserted into the disclosed metered dose inhaler port.

### DETAILED DESCRIPTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

Metered dose inhalers (MDIs) are used in the treatment of a variety of breathing conditions. These include most obviously asthma, and also COPD (chronic obstructive pulmonary disease) and other bronchial diseases. There are a number of different medications that are administered using the metered dose inhaler depending on the disease being treated and the response of the patient to the particular medication. Table 1 contains a listing of many common medications administered using a metered dose inhaler. These medications may also be delivered by different means such as through the use of a nebulizer. Table 1 also includes common dosages that are administered by each actuation of the inhaler, for each medicine.

TABLE 1

| | Amount of Drug | |
|---|---|---|
| metered dose inhaler | Per Actuation | |
| Albuterol sulfate (Ventolin, Proventil, | 90 | mcg |
| Ventolin HFA, Proventil HFA, | | |
| ProAir HFA) | | |
| Beclomethasone dipropionate (QVAR) | 40 or 80 | mcg |
| Ciclesonide (Alvesco) | 80 or 160 | mcg |
| Cromolyn sodium (Intal) | 800 | mcg |
| Flunisolide (AeroBid, AeroBid-M) | 250 | mcg |
| Flunisolide hemihydrate | 80 | mcg |
| (Aerospan HFA) | (78 | mcg delivered) |
| Fluticasone propionate (Flovent HFA) | 44, 110, or 220 | mcg |
| Fluticasone propionate/salmeterol | 45, 115, or 230 | mcg/21 mcg |
| xinafoate (Advair HFA) | | |
| Ipratropium bromide (Atrovent HFA) | 17 | mcg |
| Ipratropium bromide/albuterol sulfate | 18 | mcg/90 mcg |
| (Combivent) | | |
| Levalbuterol tartrate (Xopenex HFA) | 45 | mcg |
| Pirbuterol acetate (Maxair Autohaler) | 200 | mcg |
| Mometasone/formoterol (Dulera) | 100 or 200 | mcg/5 mcg |
| Triamcinolone acetonide (Azmacort)* | 75 | mcg |

The most common method of administering a dose of a medicine inhaled through a metered dose inhaler is through the use of the familiar “boot shaped” inhaler. This inhaler is known to everyone who has frequented a sports field or swimming pool and observed children self-administering asthma medication. In the use of this device, the user places the discharge end of the “boot” in or near the mouth. The metered dose inhaler aerosol container 20 and stem 22 are squeezed together within the plastic boot dispenser. The aerosolized medication is discharged from the dispenser towards or into the patient's mouth as the patient inhales deeply. As the stem and container are squeezed together, a meter records the number of uses of the inhaler and keeps a running total. In this way a user can determine if a refill of medication is needed. When the container is empty it may be removed from the dispenser and replaced with a full container.

While the above procedure works well for ambulatory and alert patients, it of course cannot function for unconscious or otherwise impaired patients and so these medications must be administered to them by the appropriate personnel. Particularly for those patients that are being mechanically ventilated in a hospital setting, an easy method of administering the proper medication must be used. Mechanical ventilators or respirators are used for mechanical ventilation of the lungs of a patient in a medical setting. The ventilator unit is connected to a hose set; the ventilation tubing or tubing circuit, delivering the ventilation gas to the patient. At the patient end, the ventilation tubing is typically connected to a tracheal ventilation catheter or “tube”, granting direct and secure access to the lower airways of a patient. Tracheal catheters are equipped with an inflated sealing balloon element, or “cuff”, creating a seal between the tracheal wall and tracheal ventilation tube shaft, permitting positive pressure ventilation of the lungs.

One type of tracheal catheter, an endotracheal tube (ET tube), inserted through the mouth, is generally used for a number of days before a decision is made to switch a patient to a tracheostomy tube, inserted directly into the trachea through a stoma in the tracheal wall. A closed suction catheter (also called a TRACH CARE® device) may be attached to the proximal end of the trach tube and used to remove secretions that may build up in the patient's lungs. The TRACH CARE® device also has a special port to administer medication to a patient using a metered dose inhaler.

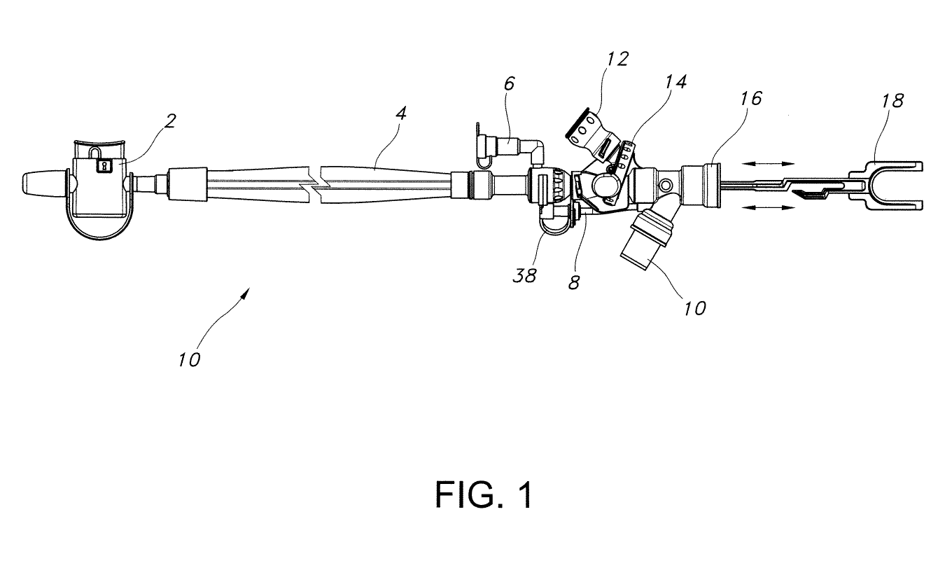

Turning now to the drawings, as illustrated in FIG. 1, there is shown a TRACH CARE® device 10 having a suction control valve 2 on its proximal end, a sheathed suction catheter 4 and a manifold 16 for connection to a trach tube (not shown). In this drawing, a shipping holder 18 is shown. The shipping holder 18 is inserted into the distal end and removed prior to the use of the device. The distal end of the device is then mated to the proximal end of a trach tube that has been installed in the patient's airway.

The manifold 16 has a ventilator port 10 for connection to a ventilator circuit for provision of air to the patient as described above. In this manifold, there is also located a rotary connection 14 so that the sheathed suction catheter 4 may be rotated out of position if desired. A seal cassette 12 may be rotated into position by the movement of the rotary connection 14 so that other devices may be inserted through the seal cassette 12 into the respiratory tract of a patient without opening the ventilator circuit. The seal cassette 12 is described in greater detail in U.S. patent application Ser. No. 12/334,067. There is a wash port 6 that connects to the distal end of the sheathed suction catheter 4 to allow for the injection of water to clean the suction catheter. The wash port 6 should not be confused with the metered dose inhaler port 36. The metered dose inhaler port 36 discharges at its distal end directly into the manifold 16 so that any medicine injected through this port 36 enters the air stream to the patient. It is important to note that this rotary manifold arrangement is not required for the disclosed metered dose inhaler port and the port can be incorporated into virtually any ventilator system.

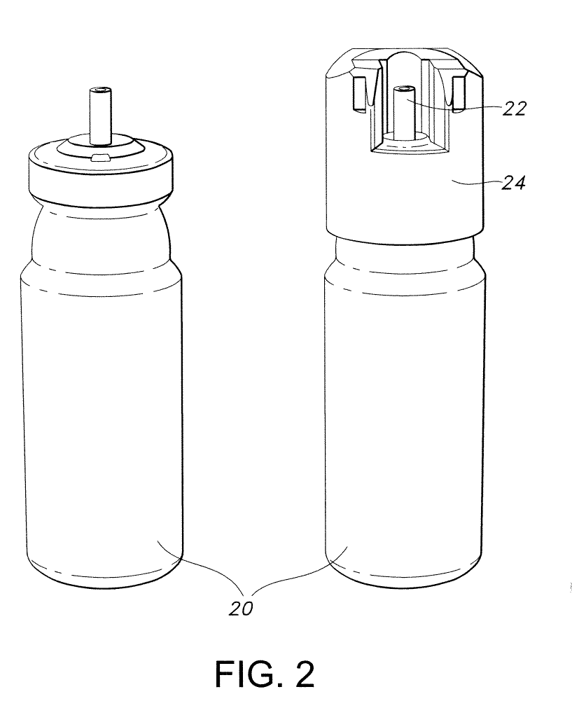

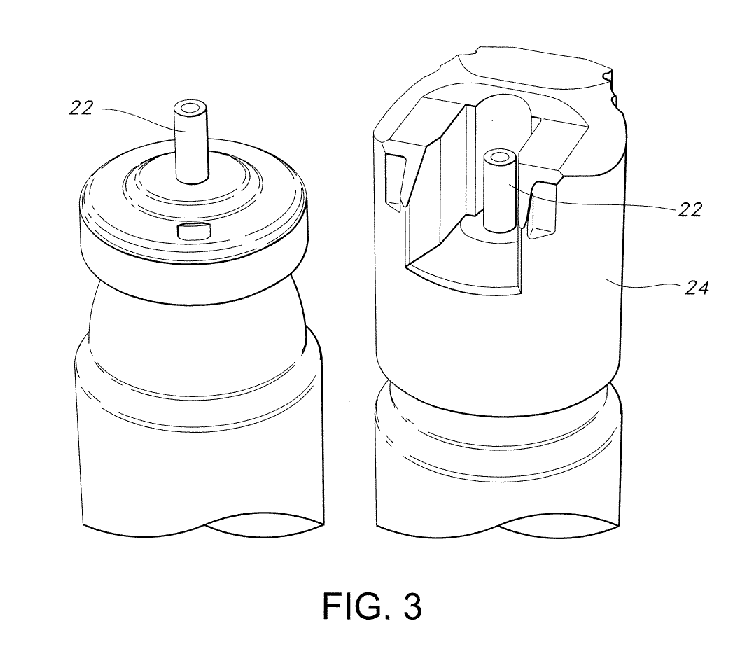

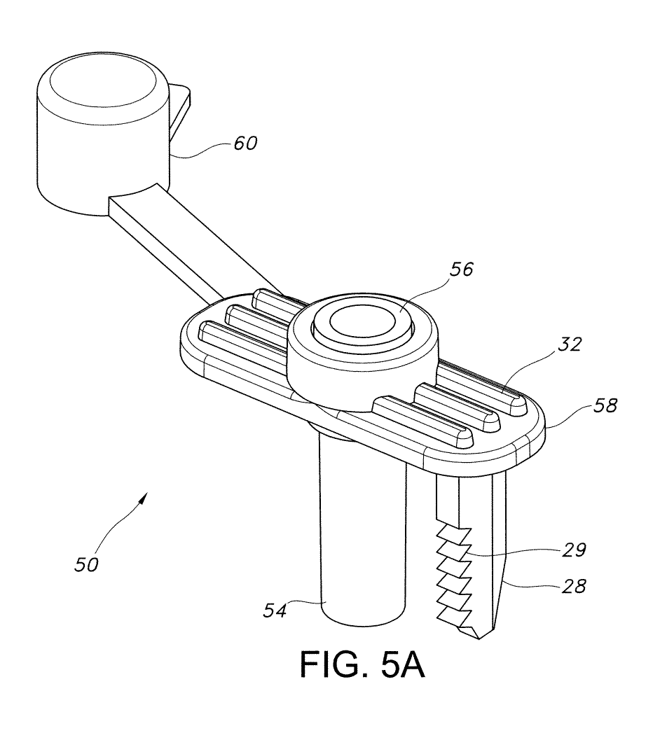

FIGS. 2 and 3 show the metered dose inhaler canister 20 without (left) and with (right) a dose counter 24. The metered dose inhaler stem 22 can be seen clearly in the left hand drawings but is somewhat obscured in the right hand drawings because of the dose counter 24. FIG. 3 also reveals the opening 26 through which the counter is accessed. The counter opening 26 of the metered dose inhaler dose counter 24 is designed to accept a prong 28 (FIG. 5A) that ratchets a small wheel totaling counter within the metered dose inhaler dose counter 24. This wheel is not visible in the views shown as it is in internal piece. The total number of doses recorded by the wheel totaling counter is generally shown using a small display on the side of the metered dose inhaler dose counter 24 opposite that shown in FIG. 3. This display is designed to be visible when the canister 20 with the dose counter 24 is inserted into a boot shaped inhaler.

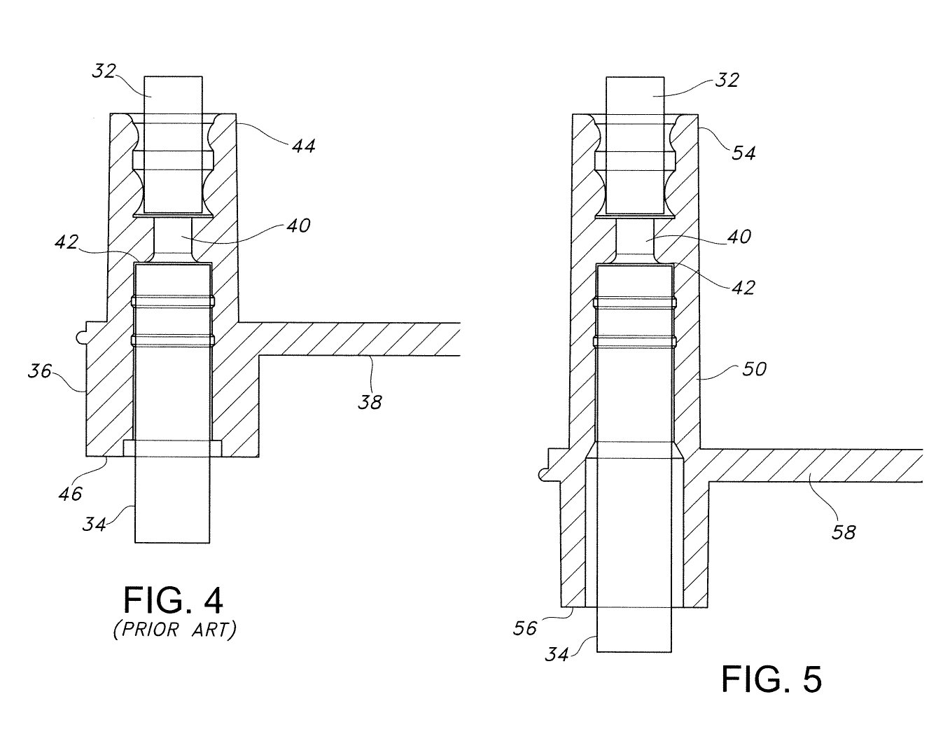

FIG. 4 shows a cross-sectional view of the prior art port 36 with a metered dose inhaler stem 32 inserted. Tubing 34 is inserted into the distal end 46 of the port 36 and leads to the manifold 16. The port 36 has a platform 38 and optionally a tether or fob that holds a cap 60 (not shown in this view) that may be used to close the port 36 on the proximal end 44 once the metered dose inhaler stem 32 is withdrawn. In use, the metered dose inhaler stem 32 is inserted into the proximal end 44 of the port 36. The user may hold the port 36 between his first two fingers using the platform 38 and place his thumb on the bottom of the metered dose inhaler canister 22, squeeze the canister 22 and port 36 together, thereby instilling medicine through the port 36, the tubing 34 and into the manifold 16. The tubing 34 has a proximal end 42 that is very close to the end of the metered dose inhaler stem 32 when it is inserted so that the drug path 40 within the port 36 is minimized.

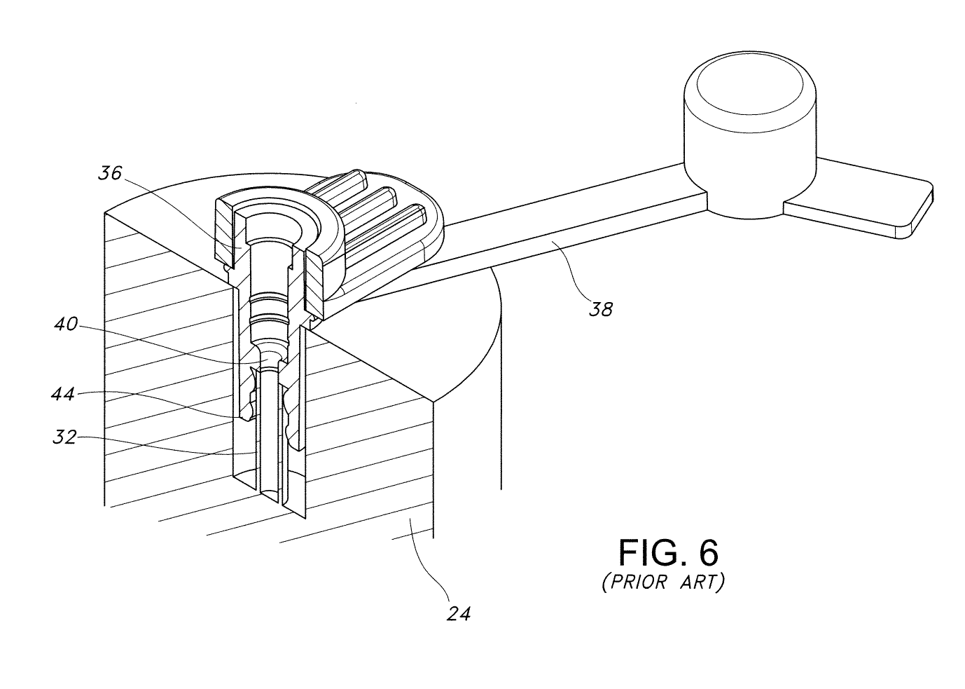

FIG. 6 illustrates the problem of inserting a metered dose inhaler stem 32 using a dose counter 24 into the prior art port 36. As can be seen, the distance from the platform 38 to the proximal end 44 is too short to allow the stem 32 to be inserted sufficiently to compress the stem 32 and port 36 together to actuate the metered dose inhaler container and discharge medicine. In this view the cap 60 is visible. The short stem 32 of the prior art device causes interference between the platform 38 and the end of the metered dose inhaler dose counter 24. This renders the newer type of metered dose inhaler container 20 with dose counter 24 unusable with the older type of metered dose inhaler ports 36.

One possible solution to the problem of mismatched metered dose inhaler ports 36 and metered dose inhaler dose counters 24 is to remove the dose counter 24 from the container 20. The dose counter 24 is an “add-on” device and not an integral part of the container 20. This is not, however, easily done. The metered dose inhaler dose counter 24 is designed to slip over the stem 22 and onto the end of the container 20 and to be locked onto the end of the container 20. Removing the dose counter 24 from the container 20 cannot generally be accomplished by users of average or even above average strength without a tool of some sort. It may be possible to wedge the end of a small screwdriver, for example, between the dose counter 24 and the container 20, and pry the dose counter 24 off of the container 20, though it appears that this may damage the container 20. Slippage of the screwdriver may further result in injury to the user so is not advised. Prying the dose counter 24 from the container 20 may also damage the stem 22 in the process so, again, is not advised.

FIG. 5 shows a cross-sectional view of the disclosed port 50 with a metered dose inhaler stem 32 inserted. The tubing 34 again is inserted into the distal end 56 of the port 50 and leads to the manifold 16. The port 50 has a platform 58 that may also hold a cap (not shown in this view) that may be used to close the port 50 on the proximal end 54 once the metered dose inhaler stem 32 is withdrawn. The tubing 34 has a proximal end 42 that is very close to the end of the metered dose inhaler stem 32 when it is inserted so that the drug path 40 in the port 50 is minimized and is in fact exactly the same as that of the prior art port 36. The method of use of the disclosed port 50 is the same as that of the prior art port 36.

FIG. 5A shows a perspective view of an alternate version of the disclosed metered dose inhaler port 50. In this view the tubing 34 is not present. This FIG. 5A has the ratcheting prong 28 that may be inserted into the metered dose inhaler dose counter opening 26. The prong 28 is aligned with the counter opening 26 as the metered dose inhaler container 20 with dose counter 24 is mated to the metered dose inhaler port 50. The metered dose inhaler prong 28 is most conveniently molded to be of a piece with the platform 58 and the rest of the port 50. It is possible, though difficult from a manufacturing point of view, to produce the prong 28 separately and connect it in some manner with the platform 58. This may be a point of weakness in the future however, and may cause a problem should it break off from the platform. When the prong 28 is inserted into the counter opening 26 and the metered dose inhaler container 20 is moved toward the port 50, the teeth 29 of the prong 28 will turn the internal dose counter wheel (mentioned above) and increase the count of doses dispensed by one. In this way, the user knows approximately when it will be necessary to acquire a new container 20. It should be noted that on the platform 58 in this view may be seen some ridges 31. The ridges 31 may be used to rest the user's fingers and squeeze the metered dose inhaler port 50 towards the container 20.

It should be noted that although the prong and tooth method of advancing the dose counter is described above (since it is the prevalent method) any other system of advancing a counter may be used, provided it is compatible with the port design. For example, a straight prong without teeth may be used in which the prong depresses a tab that advances the counter. Other means of advancing the counter are intended to be within the scope of this disclosure as they may be envisioned by one skilled in the art without undue experimentation.

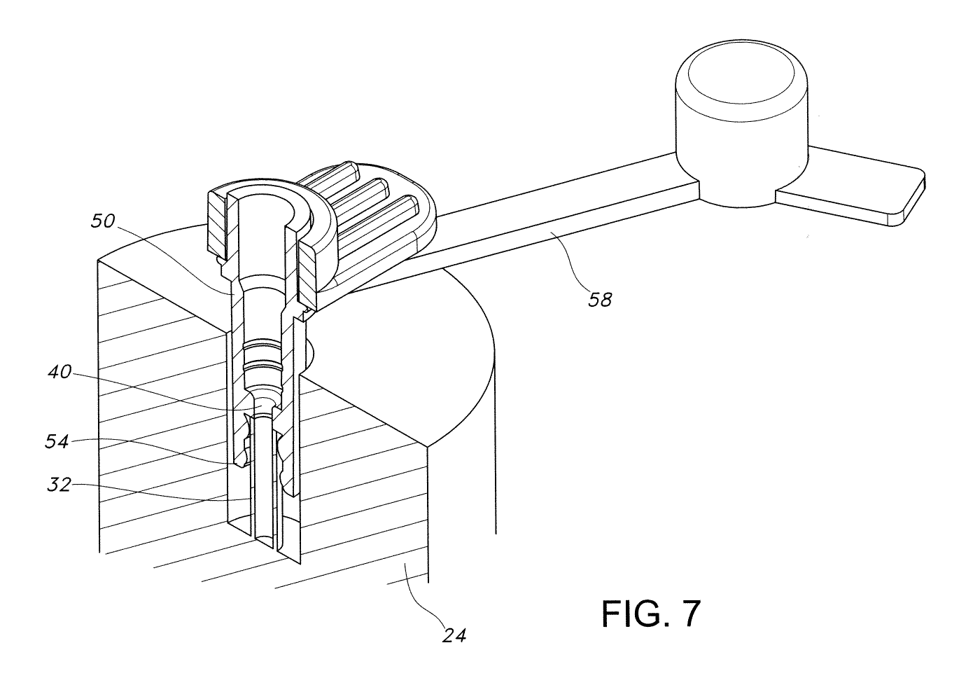

FIG. 7 shows the disclosed port 50 used with a metered dose inhaler stem 32 using a dose counter 24. As can be seen, the distance from the platform 58 to the proximal end 54 is sufficient to allow the stem 32 to be inserted to compress the stem 32 and port 50 together to actuate the metered dose inhaler container 20 and discharge medicine. The tubing 34 length in the prior art port 36 and the disclosed port 50 is the same, thus allowing the metered dose inhaler dose counter 24 to be used while maintaining the drug path 40 the same.

It is important that the drug path be unchanged when going from the prior art metered dose inhaler to the disclosed metered dose inhaler because the testing to make a change in the drug path is substantial. The US Food and Drug Administration (FDA) has guidelines for most medical products including metered dose inhalers. This advice may be found on their website under medical devices as “reviewer guidance for nebulizers, metered dose inhalers, spacers and actuators”. (http://www.fda.gov/MedicalDevices/DeviceRegulationandGuidance/GuidanceDocuments/ucm081282.htm) The specific advice for metered dose inhalers is reproduced below.

### I. Metered Dose Inhaler, Actuator, and Spacers

In addition to the particle size distribution testing for all aerosol delivery devices referenced above, the following tests should be conducted to further characterize the performance of metered dose inhaler devices, actuators, and spacers. The three drugs tested for particle size distribution should be tested as follows:
   1. The metered dose inhaler/actuator device must be directly compared to a predicate device. For example, particle size distribution data should be gathered for the predicate and new device so that a direct comparison utilizing the identical particle sizing method can be made. Particle size distributions should be collected at three different times during the life of the canister, i.e., when the drug canister is full, ½ full, and toward the end of the canister lifetime.
   2. Spray pattern and plume geometry are used to characterize primarily the performance of the valve and actuator. Spray pattern and plume geometry must be collected for the MDI/actuator assembly. Spray pattern should be determined by impingement of the spray on a thin-layer chromatography (TLC) plate. Since the observed spray pattern may vary with the distance from the actuator orifice to the TLC plate, a spray pattern profile should be determined a-t-7-a distance between 2.5 and 7.5 cm from the mouthpiece for the new device and a legally marketed predicate device. Dimensional analysis of the geometry of the plume and the distribution of particles in the plume may vary depending on the configuration of the device. Plume geometry (side view of the plume) data for the new product and the reference product are optional but highly encouraged for both products.
   3. A spacer device must be directly compared to a predicate spacer as well as directly compared to an MDI alone without the spacer attached. Particle size distribution data should be gathered for the predicate device and the new device utilizing the identical MDI attached to the devices. Furthermore, a particle size comparison between the new spacer device and the attached MDI alone must be presented. Each spacer must have particle size distribution data for each drug classification type for which it is intended (in this context drug classification type refers to the general types listed above, i.e., bronchodilators, steroids, anti-allergics, etc.). For example, data must be gathered with at least one bronchodilator, one anti-allergic, and one steroid if the spacer is intended for use with each specific drug classification type.
      In vitro data must be confirmed with small in vivo confirmational trials to assess the relative safety and effectiveness of the spacer device when compared to the MDI drug product alone. The in vivo trials must consist of at least two trials per drug classification type; one trial to assess the effectiveness and one trial for safety. The trials should directly compare the relative effectiveness and safety of the spacer to the MDI. Labeling for the spacer should be consistent with the results of the in vitro and in vivo study results.
   4. The potency or the average amount of drug delivered per spray must be specified. Potency tests should be performed for each specific drug in the claims of intended use.

The cost for such testing can be in the hundreds of thousands of dollars. If no change is made to the drug path it is unnecessary to re-test the device so this expense can be avoided. Avoiding a change in the drug path so that prior testing can be relied upon is therefore highly preferred.

In order to be successful, the platform, cap tether, or any other obstruction, must be a sufficient distance from the proximal end of the port to avoid interference with the dose counter when squeezing the metered dose inhaler container and port together to actuate the metered dose inhaler. This distance will be referred to herein as the “critical distance”. The prior art port had a critical distance of 0.330 inches (8.38 mm) between the tether 38 and the proximal end 44 of the port 36. The disclosed port 50 has a critical distance of 0.530 inches (13.46 mm). This critical distance may be greater or smaller depending on the height of the dose counter, which may vary by manufacturer. The critical distance of the disclosed metered dose inhaler port may be about 50 percent greater than prior art devices.

It should further be noted that the results throughout this document were developed using English units. In the case of any discrepancy between the Metric (SI) units and the English units herein, the English units should be regarded as the primary authority.

While it was found that the critical distance is important, it was surprisingly found that merely lengthening the port was insufficient to allow metered dose inhalers with dose counters to be used. When the port was lengthened and a metered dose inhaler with a dose counter attached inserted, the port bent and buckled to such a degree that the discharge of the metered dose inhaler was not clinically practical. It was found that the lengthened port made from the same polymer as the prior art port can bend on insertion of the metered dose inhaler stem, but definitely bends upon the application of force to actuate it if it has been successfully inserted.

Examining the polymer used to produce the port revealed that the prior art port was made from a PVC material having a durometer reading of 72 A. Using this same polymer allowed too much movement of the disclosed port. It was believed that the 72 A hardness polymer in this configuration was too soft to resist bending upon the application of force. After some experimentation, it was found that a polymer having a durometer reading of 85 A functioned well. It did not bend or buckle upon insertion or activation of the canister. Therefore, a material having a hardness durometer of greater than 72 A and more desirably at least 85 A should function well. It should be noted that most polymeric materials have a specification that could allow them to be plus or minus 5 percent of their stated value so the 72 and 85 Shore A hardness numbers have a tolerance of plus or minus 5 percent or more desirably 3 percent.

This material having a greater Shore A hardness, also desirably a PVC, though phthalate free, has sufficient stiffness to allow the insertion of the metered dose inhaler stem into the port without undue bending, buckling, twisting, kinking and/or crimping. A particularly suitable polymer is a phthalate-free polyvinylchloride (PVC) available from Colorite Polymers (Ridgefield, N.J.) under the designation 8588G-015SF.

The relative hardness of the polymers may be measured by the Shore hardness, a series of scales that is known to those skilled in the art. Hardness is measured using a device called a “durometer”; an instrument specifically developed to measure relative hardness, and is usually performed following ASTM D2240. In the Shore A and D hardness or durometer scales, a higher number indicates a polymer that is harder than a polymer having a lower number within each scale. The Shore A and D scales are used for different types of polymers. Typically the Shore A scale is used for softer, more elastic polymers and the Shore D scale used for stiffer polymers. When comparing the Shore A and Shore D scales, low D values are typically harder than high A values. For example, a 55 D hardness is typically harder than a 90 A Shore hardness value.

Bending or buckling of a material may be measured by a column buckling test. It should be noted that the stiffness of a polymer as discussed above, when made into a piece such as a metered dose inhaler port, will also be reflected in the column buckling test, so that the two can be correlated. And, while only single component polymeric pieces were tested herein, it is possible to envision equivalent structures that are not made from a single polymer and that will also function sufficiently. Such equivalent structures, for example, over-molded pieces where a softer polymer is molded around a harder material, are intended to be included in this disclosure. Other methods or achieving higher stiffness of the port like the use of sleeves, ferrules, or a blend or other combination of hard and soft materials are also intended to be included herein.

Should it be desired to use an overmolded structure or one of the other possible designs, the equivalent hardness will not be directly available. The column buckling test should be run in order to develop the proxy for the hardness by testing a piece made from at polymer with a hardness above 72 A, desirably 85 A or more, and testing the structure desired. The test should be run with a slightly eccentric load, not an axially applied load, since in practice the piece will be subject to an eccentric load when pressed by the user's fingers.

While the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. A metered dose inhaler port for a ventilated patient wherein said port has a critical distance sufficient to allow a metered dose inhaler to discharge into said port when the metered dose inhaler comprises a dose counter.

2. The port of claim 1 wherein said port has a stiffness sufficient to avoid bending of said port upon insertion of the metered dose inhaler stem.

3. The port of claim 2 wherein said port is made from a phthalate free polymer having a durometer of at least 85 A.

4. The port of claim 3 wherein said port is made from polyvinyl chloride.

5. The port of claim 1 having a critical distance of at least 13.46 mm.

6. The metered dose inhaler port of claim 5 wherein said port further comprises a means for advancing the dose counter.

7. The metered dose inhaler port of claim 6 wherein said port further comprises a prong that is inserted into an opening on the dose counter and advances the dose counter.

8. A ventilator manifold having a metered dose inhaler port, wherein said port has a critical distance of 13.46 mm plus or minus 10 percent and a durometer of above 72 A plus or minus 5 percent.

9. The manifold of claim 6 wherein said port is made from a phthalate free polyvinyl chloride.

10. A metered dose inhaler port for a ventilated patient wherein said port has a critical distance at least 50 percent greater than a prior art device and has a Shore Hardness durometer of above 72 A.

11. The metered dose inhaler port of claim 10 wherein said port further comprises a means for advancing the dose counter.

12. The metered dose inhaler port of claim 11 wherein said port further comprises a prong that is inserted into an opening on the dose counter and advances the dose counter.
